(19) United States
(12) Patent Application Publication
(10) Pub. No.: US 2012/0095445 A1
(43) Pub. Date: Apr. 19, 2012

(54) METHODS AND COMPOSITIONS FOR INCREASING TRICHOGENIC POTENCY OF DERMAL CELLS

(75) Inventors: Ying Zheng, West Chester, PA (US); Marylene Boucher, St-Ignace-de-Loyola (CA); Ying Homan, Ambler, PA (US); Charles Hollow, Scranton, PA (US); Polina Mamontov, Philadelphia, PA (US); Kurt Stenn, Princeton, NJ (US)

(73) Assignee: Aderans Research Institute, Inc.

(21) Appl. No.: 13/378,732

(22) PCT Filed: Jun. 16, 2010
(86) PCT No.: PCT/US10/38833

§ 371 Date: Dec. 16, 2011

Related U.S. Application Data
(60) Provisional application No. 61/187,894, filed on Jun. 17, 2009.
(60) Provisional application No. 61/227,540, filed on Jul. 22, 2009.

(51) Int. Cl. A61K 35/12; A61P 17/14; A61M 37/00; C12N 5/071

(52) U.S. Cl. 604/522; 435/375; 435/366; 424/93.7

(57) ABSTRACT

Methods and compositions for increasing trichogenicity of cells in culture are provided. One embodiment provides culturing dissociated mammalian dermal cells in vitro in the presence of an effective amount of one or more sonic hedgehog (Shh) pathway agonists to increase the trichogenicity of the dissociated mammalian dermal cells compared to untreated dissociated mammalian dermal cells. The cell culture optionally includes epidermal cells. Preferred Shh agonists include, but are not limited to CUR-0236715 and CUR-0201365 available from Curis, Inc. Trichogenicity is measured using the Aderans Hair Patch Assay™. The cultured dermal cells can be maintained in culture in the presence of the one or more Shh agonists for at least 1 to 7 or more days prior to harvest. The treated, cultured dermal cells can be used to treat hair loss in a mammalian subject, preferably a human, by implanting them in an effective amount to induce hair follicle formation.

### CROSS-REFERENCE TO RELATED APPLICATIONS

This application claims benefit of and priority to U.S. Provisional Patent Application No. 61/187,894 filed Jun. 17, 2009 and U.S. Provisional Patent Application No. 61/227,540 filed Jul. 22, 2009, and where permissible both of which are incorporated by reference in their entirety.

### FIELD OF THE INVENTION

The invention is generally directed to methods and compositions for increasing trichogenic potency of dermal cells, in particular methods and compositions for activating or stimulating the Sonic Hedgehog pathway.

### BACKGROUND OF THE INVENTION

Hair loss or alopecia is a common problem in both males and females regardless of their age. There are several types of hair loss, such as androgenetic alopecia, alopecia greata, telogen effluvium, hair loss due to systemic medical problems, e.g., thyroid disease, adverse drug effects and nutritional deficiency states as well as hair loss due to scalp or hair trauma, discoid lupus erythematosus, lichen planus and structural shaft abnormalities. (Hogan and Chamberlain, South Med J., 93(7):657-62 (2000)). Androgenetic alopecia is the most common cause of hair loss, affecting about 50% of individuals who have a strong family history of hair loss. Androgenetic alopecia is caused by three interdependent factors: male hormone dihydrotestosterone (DHT), genetic disposition and advancing age. In the genetically susceptible host DHT causes hair follicles to miniaturize, resulting in weak hairs and to shorten the anagen phase of the hair follicle growth cycle. Over time, large hair shafts are shed and they are replaced by very short, thin shafts giving the impression of massive hair loss.

Possible options for the treatment of alopecia include hair prosthesis, surgery and topical/oral medications. (Hogan & Chamberlain, 2000; Bertolino, J Dermatol, 20(10):604-10 (1993)). While drugs such as minoxidil, finasteride and dutasteride represent significant advances in the management of male pattern hair loss, the fact that their action is temporary and the hairs are lost after stopping therapy continues to be a major limitation (Bouhanna, Dermatol Surg, 29(11):1130-1134 (2003); Avram, et al., Dermatol Surg, 28:894-900 (2002)). In view of this, surgical hair restoration and tissue engineering may be the only permanent methods of treating pattern baldness. The results from surgical hair transplantation can vary. Early donor punch techniques often resulted in a highly unnatural “doll hair look” or “paddy field look” over the recipient area. Although advances have been made in surgical hair transplantation, for example, single follicle hair grafts or 1 mm punches, the procedures are time consuming and costly and most important to this application, the number of donor follicles on a given patient is limited.

Tissue/cell engineering to treat hair loss includes transplanting cells into an area to induce hair follicle formation and subsequent hair shaft formation. Theoretically, tissue/cell engineering may be employed to treat hair loss due to a variety of diseases, syndromes, and injuries. Hair follicle induction and growth involves active and continuous epithelial and mesenchymal interactions (Stenn & Paus, Physiol Reviews, 81:449-494, (2001)). In the embryo, the first hair follicles grow from a thickening of the primitive epidermis which is controlled by finely tuned signals arising in the epidermis itself and the underlying dermis. Early studies (Cohen, J Embryol Exp Morphol, 9:117-127 (1961)) using adult rodent hair follicles showed that the dissected deep mesenchymal portion of the hair follicle, the follicular or dermal papilla, when implanted under adult epidermis, will induce new hair follicles. This powerful tissue induction is ascribed to a unique property of the cells in the papilla and the dermal sheath (McElwee et al., J Invest Dermatol, 121:1267-1275 (2003)).

Multiple studies have shown that hair follicle morphogenesis (Wang et al., J Invest Dermatol, 114:901-908 (2000); Mill et al., Genes Dev, 17:282-294 (2003); Lehman, J., et al., J Invest Dermatol, 129:438-448 (2009); St Jacques, B., Current Biology, 8:1058-1068 (1998) and progression of the hair cycle (Oro, A. E., & Higgins, K., Dev Biol, 255:238-248 (2003); Paladini, R. D., et al., J Invest Dermatol, 125:638-646 (2005); Sato, N., et al., J Clin Invest, 104:855-864 (1999); Sato, N., et al. J Natl Cancer Inst, 93:1858-1864 (2001)) depend on sonic hedgehog (Shh) signaling. When the Shh pathway is blocked (using an antibody), hair follicle formation does not occur (Wang, L. C., et al., J Invest Dermatol, 114:901-908 (2000)). When intact Shh is injected into telogen skin, anagen hair growth is initiated (Sato, N., et al., J Clin Invest, 104:855-864 (1999)). The latter finding also occurs when synthetic Shh agonists are used (Paladini, R. D., et al., J Invest Dermatol, 125:638-646 (2005)).

Elements of the Shh pathway are expressed in both the epithelial and dermal components. Because dermal cells are instrumental to hair follicle morphogenesis and cycling, it is an object of the invention to provide methods and compositions for increasing trichogenicity of dermal cells.

It is another object of the invention to provide methods and compositions for treating hair loss in a subject.

It is still another object of the invention to provide methods for increasing or maintaining trichogenicity of dermal cells in culture.

It is yet another object of the invention to provide methods and compositions for activating or stimulating the Shh signal transduction pathway to increase or maintain trichogenicity of dermal cells.

It is another object of the invention to provide methods and compositions for producing trichogenic dermal cells for inducing hair follicles in a subject.

### SUMMARY OF THE INVENTION

Methods and compositions for increasing trichogenicity of cells in culture are provided. One embodiment provides culturing dissociated mammalian dermal cells in vitro in the presence of an effective amount of one or more sonic hedgehog (Shh) pathway agonists. The agonist can interact at any point in the pathway to activate the signal transduction pathway to increase the trichogenicity of the dissociated mammalian dermal cells compared to untreated dissociated mammalian dermal cells. For example, the agonist can be to Smoothened, the signal tranducer of Shh pathway. The cell culture optionally includes epidermal cells. Preferred Shh pathway agonists or Smoothened agonists include, but are not limited to, CUR-0201365 and CUR-0236715 available from Curis, Inc. Trichogenicity is measured using the Aderans Hair Patch Assay™. The cultured dermal cells can be maintained in culture in the presence of the one or more Shh pathway agonists for at least 1, 2, 3, 4, 5, 6, 7 or more days prior to harvest.

The treated dermal cells can be used to treat hair loss in a mammalian subject, preferably a human. Typically, an explant of skin tissue is obtained from a subject to be treated. The explant is treated to dissociate the cells into a suspension of cells which are then cultured in the presence of one or more Shh pathway agonists. In one embodiment, the cells are cultured for at least seven days prior to implanting the cells into the skin of the subject. Preferably the cells are autologous, but it will be appreciated that the cells can be allogenic. An effective amount of cultured dermal cells are implanted in the skin of the subject to form or induce the formation of a hair follicle.

Another embodiment provides an isolated population of dermal cells having at least 1, 5, 10, 15, 20, 25, 30% up to 300% increased trichogenicity as determined by the Aderans Hair Patch Assay™ compared to non-treated cells. Treated cells have about 3 times the number of hair follicles in the Aderans Hair Patch Assay™ compared to same amount of non-treated cells.

In some instances, trichogenicity of cultured dermal cells has been observed to decrease when the cells are maintained in culture. It has been discovered that the amount of reduction in trichogenicity can be reduced or prevented by culturing the cells in the presence of an effective amount of one or more Shh pathway agonists. Preferably trichogenicity levels in dermal cells in culture are maintained after at least 1, 2, 3, 4, 5, 6, 7, 8, 9, or 10 passages.

### BRIEF DESCRIPTION OF THE DRAWINGS

FIG. 1 is a graph showing fold change in expression of h-GLi1 and h-PTCH1 in dermal cells treated with sonic hedgehog (Shh) pathway agonist B (CUR-0236715). “P” refers to cell passage. The cells are grown for a period of time in one dish. When the cells are transferred to a second dish the cells are considered to be passed. The first plating of cells is considered to be passage zero (P0). When the cells are lifted from the dish and passed to a new vessel the passage is P1, etc. The length of time cells have been in culture is often described by passage number.

FIG. 2A is a graph showing number of hair follicles produced in the Aderans Hair Patch Assay™ by dermal cells treated with Agonist A (CUR-201365). FIG. 2B is a graph showing number of hair follicles produced in a hybrid patch assay by dermal cells treated with the indicated amount of a Shh pathway agonist after one passage in culture. FIG. 2C is a graph showing number of hair follicles produced in the Aderans Hair Patch Assay™ by dermal cells treated with the indicated amount of a Shh pathway agonist after two passages in culture. FIG. 2D is a graph showing number of hair follicles produced in the Aderans Hair Patch Assay™ by dermal cells treated with the indicated amount of a Shh pathway agonist after three passages in culture. FIG. 2E is a graph showing number of hair follicles produced in the Aderans Hair Patch Assay™ by dermal cells treated with the indicated amount of a Shh pathway agonist after four passages in culture.

FIG. 3A is a graph showing the population doubling time of dermal cells treated with the indicated amount of a Shh pathway agonist. FIG. 3B is a graph showing number of cells (millions) of dermal cells treated with Shh pathway agonist at confluence.

FIG. 4A is a graph showing the average hair follicle number formed in the hybrid patch assay using dermal cells treated with the indicated amount of Shh pathway agonist. FIG. 4B is a graph showing the average hair follicle number formed in the Aderans Hair Patch Assay™ using dermal cells treated with the indicated amount of Shh pathway agonist after two passages in culture.

FIG. 5A is a graph showing the average number of cells (millions) in flasks using dermal cells treated with the indicated amount of Shh pathway agonist after one passage in culture. FIG. 5B is a graph showing the average number of cells (millions) in flasks using dermal cells treated with the indicated amount of Shh pathway agonist after two passages in culture. FIG. 5C is a graph of population doubling time (days) for dermal cells treated with the indicated amount of Shh pathway agonist after one passage in culture. FIG. 5D is a graph of population doubling time (days) for dermal cells treated with the indicated amount of Shh pathway agonist after two passages in culture.

FIG. 6A is a graph showing the average number of hair follicles formed in the Aderans Hair Patch Assay™ using dermal cells treated continuously for a short time (7 days before harvest) with the indicated amount of a Shh pathway agonist after one passage in cell culture. FIG. 6B is a graph showing the average number of hair follicles formed in the Aderans Hair Patch Assay™ using dermal cells treated continuously for a short time (7 days before harvest) with the indicated amount of a Shh pathway agonist after two passages in cell culture.

FIG. 7 is a graph of the number of hair follicles formed from human fetal cell cultures in the Aderans Hair Patch Assay™ versus the indicated cell culture passage P0, P1, P2 or P3 in cells culture with a Shh pathway agonist. Each passage has a pair of columns with the left column corresponding to results obtained without treating the cells with an agonist and a right column showing results with cells treated with an agonist.

FIG. 8 is a graph of number of hair follicles formed from human fetal cell cultures in the Aderans Hair Patch Assay™ in untreated or treated cells at the indicated cell culture passage.

FIG. 9 is a graph of the number of hair follicles formed from human fetal cell cultures in the Aderans Hair Patch Assay™ in the indicated medium.

### DETAILED DESCRIPTION OF THE INVENTION

### Definitions

The term “trichogenic cells” refers to cells that induce hair follicle formation when administered to the skin of a subject.

As used herein the term “isolated” is meant to describe cells that are in an environment different from that in which the cells naturally occur e.g., separated from its natural milieu such as by separating dermal cells from skin explant.

The terms “individual”, “host”, “subject”, and “patient” are used interchangeably herein, and refer to a mammal, including, but not limited to, murines, simians, humans, mammalian farm animals, mammalian sport animals, and mammalian pets.

As used herein the term “effective amount” or “therapeutically effective amount” means an amount of cells sufficient to induce hair follicle formation or to induce vellus hair to form terminal hair or to induce a miniaturizing hair to reverse this process and become a terminal hair. With regard to cell culture, an “effective amount” of a hedgehog agonist refers to an amount of the agonist applied as part of an in vitro culture protocol for dermal cells that increases or maintains the trichogenicity of the cultured dermal cells. Preferred dermal cells include, but are not limited to, dermal cells. The cells are preferably mammalian cells, more preferably human cells.

The term “skin” refers to the outer protective covering of the body, including the corium, epidermis, and dermis and is understood to include sweat and sebaceous glands, as well as hair follicle structures.

The term “hedgehog agonist” or “sonic hedgehog agonist” refers to an agent which potentiates or recapitulates the bioactivity of hedgehog, such as to activate transcription of target genes.

As used herein, “dermal cells” or “a population of dermal cells” refers to a population of cells that contains at least 30%, 40%, 50%, 60%, 70%, 80%, 90%, 95%, 97%, 98%, 99% or 100% dermal cells. Methods for identifying a cell as a dermal cell are known in the art.

As used herein, “epidermal cells” or “a population of epidermal cells” refers to a population of cells that contains at least 30%, 40%, 50%, 60%, 70%, 80%, 90%, 95%, 97%, 98%, 99% or 100% epidermal cells. Methods for identifying a cell as an epidermal cell are known in the art.

### II. Hedgehog Signaling

The Drosophila melanogaster Hedgehog mutant was so named because the phenotype of this mutant had prominent epidermal spikes in larval segments that normally are devoid of these extensions. In mammals, the Hedgehog (Hh) protein family of secreted glycoproteins includes at least three members: Sonic Hedgehog (Shh), Desert Hedgehog (Dhh), and Indian Hedgehog (Ihh).

Hedgehog (Hh) proteins are morphogens in many tissues during embryonic development and are important mediators of intercellular signaling. The Hedgehog pathway is important in regulating cell patterning, differentiation, proliferation, survival and growth in the embryo and the adult Vertebrate Hedgehog proteins are crucial to a number of epithelial-mesenchymal inductive interactions during neuronal development, limb development, lung, bone, hair follicle and gut formation.

Signaling in the Hh pathway begins with the binding of the Shh to its receptor Patched (Ptc) a 12-transmembrane domain protein. The mammalian genome contains 2 patched genes, ptc1 and ptc2. In the absence of Shh, Ptc suppresses the activity of the seven-transmembrane protein Smoothened (Smo). When Shh is present and binds to Ptc, the repression of Smo is suspended and leads to the activation of fused (Fu), a serine-threonine kinase, and the disassociation of a zinc finger transcription factor of the mammalian Gli family (corresponding to Ci in Drosophila), from the microtubule-associated Fu-Gli-Su(fu) complex [Su(fu): Suppressor of Fused]. Gli transcription factors include Gli1, Gli2, and Gli3. Shh inhibits repressor formation by Gli3, but not by Gli2. These transcription factors translocate to the nucleus and induce target gene transcription. Members of the pathway including Gli1 and Ptc1 are themselves transcriptional targets.

A. Sonic Hedgehog

Shh is involved in pattern formation of vertebrate organs including brain, heart, lung, skeleton, and skin (Sato, N. et al., J Clincal Investigation, 104(7):855-864). In skin, Shh is required for hair follicle morphogenesis during embryogenesis and for regulating follicular growth and cycling in the adult (Paladini, R. D., et al., J Invest Dermatol, 125:638-646 (2005)). Transient overexpression of Shh in postnatal mouse skin initiates the onset of anagen growth phase of hair follicles. (Sato, N. et al. J Clincal Investigation, 104(7):855-864); Sato, N. et al., J. National Cancer Inst., 93(24):1858-1864 (2001).

The expression of Shh starts shortly after the onset of gastrulation in the presumptive midline mesoderm, the node in the mouse, the rat and the chick, and the shield in the zebrafish. In chick embyros, the Shh expression pattern in the node develops a left-right asymmetry.

In the CNS, Shh from the notochord and the floorplate appears to induce ventral cell fates. When ectopically expressed, Shh leads to a ventralization of large regions of the mid- and hindbrain in mouse, Xenopus and zebrafish. In explants of intermediate neuroectoderm at spinal cord levels, Shh protein induces floorplate and motor neuron development with distinct concentration thresholds, floor plate at high and motor neurons at lower concentrations. Moreover, antibody blocking suggests that Shh produced by the notochord is required for notochord-mediated induction of motor neuron fates. High concentration of Shh on the surface of Shh-producing midline cells appears to account for the contact-mediated induction of floorplate observed in vitro, and the midline positioning of the floorplate immediately above the notochord in vivo. Lower concentrations of Shh released from the notochord and the floorplate presumably induce motor neurons at more distant ventrolateral regions in a process that has been shown to be contact-independent in vitro. In explants taken at midbrain and forebrain levels, Shh also induces the appropriate ventrolateral neuronal cell types, dopaminergic and cholinergic precursors, respectively, indicating that Shh is a common inducer of ventral specification over the entire length of the CNS. These observations raise a question as to how the differential response to Shh is regulated at particular anteroposterior positions.

Shh from the midline also patterns the paraxial regions of the vertebrate embryo, the somites in the trunk and the head mesenchyme rostral of the somites. In chick and mouse paraxial mesoderm explants, Shh promotes the expression of sclerotome specific markers like Paxl and Twist, at the expense of the dermamyotomal marker Pax3. Moreover, filter barrier experiments suggest that Shh mediates the induction of the sclerotome directly rather than by activation of a secondary signaling mechanism. Shh also induces myotomal gene expression, although some experiments indicate that members of the WNT family, vertebrate homologues of Drosophila wingless, are required in concert.

B. Indian Hedghog

Ihh plays an important role in the regulation of chondrogenic development. During cartilage formation, chondrocytes proceed from a proliferating state via an intermediate, prehypertrophic state to differentiated hypertrophic chondrocytes. Ihh is expressed in the prehypertrophic chondrocytes and initiates a signaling cascade that leads to the blockage of chondrocyte differentiation. Its direct target is the perichondrium around the Ihh expression domain, which responds by the expression of Gli and Pte. Most likely, this leads to secondary signaling resulting in the synthesis of parathyroid hormone-related protein (PTHrP) in the periarticular perichondrium. PTHrP itself signals back to the prehypertrophic chondrocytes, blocking their further differentiation. At the same time, PTHrP represses expression of Ihh, thereby forming a negative feedback loop that modulates the rate of chondrocyte differentiation.

C. Desert Hedghog

Desert Hedgehog (Dhh) is the most restricted in terms of expression, and Dhh null mice are viable; it is expressed primarily in the testes, both in mouse embryonic development and in the adult rodent and human. The importance of the Dhh gene and its murine homologue regarding male sex differentiation has been demonstrated in various studies (Bitgood and McMahon, Dev Biol, 172:126-138 (1995); Bitgood, M. J., et al., Curr Biol, 6:298-304 (1996)). Clark, A. M., et al., Biol Reprod, 63:1825-1838 (2000) reported that the majority of Dhh null male mice developed into pseudohermaphrodites. Other studies have demonstrated that the differentiation of peritubular myoid cells and the consequent formation of testis cords is regulated by Dhh (Pierucci-Alves, F., et al., Biol Reprod, 65:1392-1402 (2001)). Furthermore, it has been suggested that Dhh/Patched 1 signaling is a positive regulator of the differentiation of steroid-producing Leydig cells in the fetal testis (Yao, H., et al., Genes Dev, 161433-1440 (2002)). In studies in humans, Umehara, F., et al., Am J Hum Genet, 67:1302-1305 (2000) reported a homozygous missense mutation of the Dhh gene, in one patient with 46,XY partial gonadal dysgenesis associated with minifascicular neuropathy; likewise, a homozygous mutation in the Dhh gene in three patients with 46,XY complete pure gonadal dysgenesis (PGD) has been reported (Canto, P., et al., J Clin Endocrinol Metab, 89:4480-4483 (2004)). All of these findings have demonstrated that Dhh is a key molecule that intervenes in male gonadal differentiation.

### II. Agonists of Sonic Hedgehog Signal Transduction Pathway

Agonists of the sonic hedgehog signal transduction pathway are known in the art. U.S. Pat. No. 6,683,108 to Baxter et al., incorporated by reference in its entirety, discloses small molecule, non-peptidyl agonists of Shh. Agonists of the sonic hedgehog signaling pathway are also commercially available from Curis, Inc. (Cambridge, Mass.). Preferred agonists of Shh pathway include, but are not limited to, CUR-0236715 and CUR-0201365. The general structure of the agonists is provided in U.S. Pat. No. 6,683,108. Preferred agonists have the following structures:

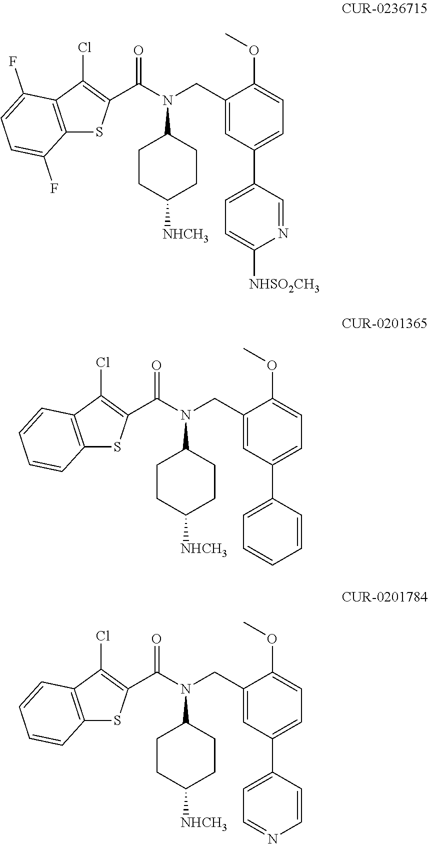

### III. Use of Agonists of Sonic Hedgehog in Cell Culture

A. Increasing Trichogenicity Cell Culture

One embodiment provides a method for increasing trichogenicity of cells in culture by culturing dissociated mammalian dermal cells in vitro in the presence of an effective amount of a sonic hedgehog pathway agonist to increase the trichogenicity of the dissociated mammalian dermal cells compared to untreated dissociated mammalian dermal cells. Preferably, the cultured cells are human dermal cells. In one embodiment, the Shh pathway agonist is present in the range of 0.125 μg/ml to 0.625 μg/ml. Another embodiment provides an isolated population of dermal cells having at least 1, 5, 10, 15, 20, 25, or 30% increased trichogenicity as determined by the hair patch assay compared to non-treated cells.

Populations of dermal cells, preferably derived from explant tissue, can be perpetuated in vitro and their trichogenicity can be increased or maintained compared to non-treated cells by contact with one or more Shh pathway agonists described above. In certain embodiments, a combination of dermal and epidermal cells are co-cultured. Preferred Shh pathway agonists include, but are not limited to, CUR-0236715 and CUR-0201365, available from Curis, Inc. (Cambridge Mass.). Generally, a method is provided including the steps of isolating dermal cells from a mammal, perpetuating these cells in vitro, preferably in growth medium including growth factors, nutrients, cofactors and other conventional cell culture additives and or supplements known in the art. Explant tissue is obtained from a subject, preferably a human. The explant tissue is typically an explant of skin containing hair follicles. The explant can be autologous or allogenic.

Cells from the explant or donor tissue are dissociated into individual cells or aggregates containing small numbers of cells. Dissociation can be obtained using any known procedure, including treatment with enzymes such as trypsin and collagenase, or by using physical methods of dissociation such as with a blunt instrument or by mincing with a scalpel to a allow outgrowth of specific cell types from a tissue.

Dissociated cells can be placed into any known culture medium capable of supporting cell growth, including MEM, DMEM, and RPMI, F-12, containing supplements which are required for cellular metabolism such as glutamine and other amino acids, vitamins, minerals and useful proteins such as transferrin. Medium may also contain antibiotics to prevent contamination with yeast, bacteria and fungi such as penicillin, streptomycin, and gentamicin. In some cases, the medium may contain serum derived from bovine, equine, chicken. A particularly preferable medium for cells is a mixture of DMEM and F-12.

Conditions for culturing should be close to physiological conditions. The pH of the culture media should be close to physiological pH, preferably between pH 6-8, more preferably close to pH 7, even more particularly about pH 7.4. Cells should be cultured at a temperature close to physiological temperature, preferably between 30° C.-40° C., more preferably between 32° C.-38° C., and most preferably between 35° C.-37° C.

Cells can be grown in suspension or on a substrate. In the case of propagating, referred to as splitting or passaging suspension cells, the cells are harvested and centrifuged at low speed. The medium is aspirated, the cells resuspended in a small amount of medium with growth factor, and the cells mechanically dissociated and resuspended in separate aliquots of cell culture medium.

Cell suspensions in culture medium are supplemented with any growth factor which allows for the proliferation of the cells and seeded in any receptacle capable of sustaining cells, preferably in culture flasks or roller bottles. Cells typically proliferate within 3-4 days in a 37° C. incubator, and proliferation can be reinitiated at any time after that by dissociation of the cells and resuspension in fresh medium containing growth factors. In a preferred embodiment, the dermal cells are cultured in the presence of one or more Shh agonist for 1, 2, 3, 4, 5, 6, 7 or more days prior to harvest.

B. Maintaining Trichogenicity in Culture

The trichogenicity of dermal cells can be maintained in culture by incorporating one or more Shh agonist into the cell culture medium. In one embodiment, the dermal cells maintain their trichogenicity after at least one, two, three, or four passages in cell culture compared to non-treated cells. In practice, there are only a certain number of cells that can be effectively grown in a given flask before they are no longer functional or exhaust the growth media and begin to die. Once a flask has reached its capacity, its cell population is split into multiple flaks and sub-cultured. This process is called passaging. The point of passage can be determined subjectively or empirically. On a laboratory scale culture flasks are visually inspected to assess the area of the plate covered, cell connectivity and cell distribution.

In one embodiment, trichogenicity levels in dermal cells in culture are maintained after at least 1, 2, 3, 4, 5, 6, 7, 8, 9, or 10 passages by culturing the cells in the presence of an effective amount of one or more Shh pathway agonist. Maintaining the trichogenicity of the dermal cells means that at least 5%, 10%, 15%, 20%, 25%, 30%, or 35% of the culture cells retain the ability to form or induce the formation of a hair follicle when implanted into the skin of a subject.

In still another embodiment, the trichogenicity of the dermal cells is increased by at least 5%, 10%, 15%, or 20% during cell culture compared to dermal cells cultured in the absence of Shh pathway agonists.

C. Measuring Tricbogenicity

Trichogenic activity of populations of dermal cells can be determined by using the Aderans Hair Patch Assay™ (Zheng, Y., J Invest Dermatol, 124: 867-876 (2005)). In this assay dissociated dermal and epidermal cells are implanted into the dermis or the subcutis of an immunoincompetent mouse. Using mouse newborn skin cells, new hair follicles typically form in this assay within 8 to 10 days. The newly formed follicle manifests normal hair shafts, mature sebaceous glands, and a natural hair cycle. Although normal cycling hair follicles are formed in this assay, the assay primarily measures the ability of cells or combinations of cells to form new follicles. Mouse dermal cells are assayed in conjunction with mouse neonatal epidermal cells.

IV. Methods of Using Dermal Cells with Enhanced Trichogenicity

A. Hair Follicle Induction

Dermal cells with increased trichogenic activity obtained using the disclosed methods may be used to generate new hair follicles in a subject. Subjects to be transplanted with dermal cells with increased trichogenic ability include any subject that has an insufficient amount of hair or an insufficient rate of hair growth. Suitable subjects include those with androgenetic alopecia, a scar of any cause, alopecia greata, telogen effluvium, thyroid disease, nutritional deficiencies, discoid lupus erythematosus, lichen planus, genetic pattern baldness or with hormonal disorders that decrease hair growth or cause loss of hair. Subjects may have these conditions or be at risk for the development of these conditions, based on genetic, behavioral or environmental predispositions or other factors. Other suitable subjects include those that have received a treatment, such as chemotherapy, or radiation that causes a decrease in hair growth or a loss of hair. Other suitable subjects include subjects that have suffered scalp or hair trauma, have structural hair shaft abnormalities, or that have had a surgical procedure, such as a skin graft, which results in an area of skin in need of hair growth. Other suitable subjects include those with a skin scar in an area where hair would be preferred. The scar may result from trauma, burn, surgery, radiation, genetic abnormality, congenital loss, etc.

Dermal and optionally epidermal cell populations may be implanted into the subject in an area where increased hair growth is desired. Preferred locations for implantation include the subject's scalp, face or eyebrow area.

The cells that are implanted into the subject may be autologous, allogenic or xenogenic. In one embodiment, dermal and epidermal cells are obtained from skin sections from a single allogenic donor or are autologous. In another embodiment, dermal and epidermal cells are obtained from skin sections from more than one donor. For example, dermal cells may be derived from one donor and epidermal cells from another donor. In a preferred embodiment, the cells that are implanted are autologous.

Dermal and epidermal cells are optionally combined at an appropriate ratio prior to implanting into the subject. Suitably, the ratio of epidermal cells to dermal cells is in the range of about 0:1, 1:1, 1:2, or 1:10. Dermal and epidermal cells may be further combined with additional cell types, such as melanocytes, fat cells, pre-adipocytes, endothelial cells, and bone marrow cells prior to implantation. The dermal and epidermal cells to be implanted may be subjected to physical and/or biochemical aggregation prior to implanting to induce and/or maintain aggregation of the cells within the transplantation site. For example, the cells can be aggregated through centrifugation of the culture. Additionally, or alternatively, a suitable aggregation enhancing substance may be added to the cells prior to, or at the time of, implantation. Suitable aggregation enhancing substances include, but are not limited to, glycoproteins such as fibronectin or glycosaminoglycans, dermatan sulfate, chondroitin sulfates, proteoglycans, heparin sulfate and collagen.

The cells may be implanted into a subject using routine methods known in the art. Various routes of administration and various sites can be used. For example, the cells can be introduced directly between the dermis and the epidermis of the outer skin layer at a treatment site. This can be achieved by raising a blister on the skin at the treatment site and introducing the cells into fluid of the blister. The cells may also be introduced into a suitable incision extending through the epidermis down into the dermis. The incision can be made using routine techniques, for example, using a scalpel or hypodermic needle. The incision may be filled with cells generally up to a level in direct proximity to the epidermis at either side of the incision. In a preferred embodiment, the cells are delivered using a device as described in US Patent Application Publication No. 2007/0233038 to Pruitt, et al.

The dosage of cells to be injected is typically between about one million to about four million cells per square cm.

In another embodiment, a plurality of small recipient sites, for example, 10, 50, 100, 500 or 1000 or more is formed in the skin into which the cells are transplanted. Each perforation can be filled with a plurality of cells. The size and depth of the perforations can be varied. The perforations in the skin can be formed by routine techniques and can include the use of a skin-cutting instrument, e.g., a scalpel or a hypodermic needle or a laser (e.g., a low power laser). Alternatively, a multiple-perforation apparatus can be used having a plurality of spaced cutting edges formed and arranged for simultaneously forming a plurality of spaced perforations in the skin. The cells can be introduced simultaneously into a plurality of perforations in the skin.

The epidermal cells, dermal cells, or combinations thereof can be combined with a pharmacologically suitable carrier such as saline solution or phosphate buffered saline solution. In a preferred embodiment the carrier is a suitable culture medium, such as Dulbecco's Phosphate Buffered Saline (“DPBS”), DMEM, D-MEM-F-12 or HYPOTHERMOSOL-FRS. The cells may also be combined with preservation solution such as a solution including, but not limited to, distilled water or deionized water, mixed with potassium lactobionate, potassium phosphate, raffinose, adenosine, allopurinol, pentastarch prostaglandin El, nitroglycerin, and/or N-acetyleysteine into the solution. Suitably, the preservation solution employed may be similar to standard organ and biological tissue preservation aqueous cold storage solutions such as HYPOTHERMOSOL-FRS.

The cells and the carrier may be combined to form a suspension suitable for injection. Each opening is implanted with an effective amount of cells to generate a new hair follicle in that opening. The number of cells introduced into each opening can vary depending on various factors, for example, the size and depth of the opening and the overall viability and trichogenic activity of the cells. The dosage of cells to be injected is typically between about one million to about 4 million cells per square cm. In one embodiment about 50,000 to about 2,000,000 cells are delivered per injection. The cell concentration can be about 5,000 to about 1,000,000 cells/μl, typically about 50,000 cells/μl to about 75,000 cells/μl. A representative volume of cells delivered per injection is about 1 to about 10 μl, preferably about 4 μl. In one embodiment, 1 to 100 injections per cm², typically 1 to 30 injections per cm² are made in the skin, preferably the scalp.

The use of dermal and/or epidermal cells derived from an allogenic source may require administration of an immunosuppressant, alteration of histocompatibility antigens, or use of a barrier device to prevent rejection of the implanted cells. Cells can be administered alone or in conjunction with a barrier or agent for inhibiting or reducing immune responses against the transplanted cells in a recipient subject. For example, an immunosuppressive agent can be administered to a subject to inhibit or interfere with normal response in the subject. The immunosuppressive agent can be an immunosuppressive drug that inhibits T cell/or B cell activity in the subject. Examples of immunosuppressive drugs are commercially available (e.g., cyclosporin). An immunosuppressive agent, e.g., drug, can be administered to a subject at a dosage sufficient to achieve the desired therapeutic effect (e.g., inhibition of rejection of the cells).

The immunosuppressive agent can also be an antibody, an antibody fragment, or an antibody derivative that inhibits T cell activity in the subject. Antibodies capable of depleting or sequestering T cells can be, e.g., polyclonal antisera, e.g., anti-lymphocyte serum; and monoclonal antibodies; e.g., monoclonal antibodies that bind to CD2, CD3, CD4, CD8 or CD40 on the T cell surface. Such antibodies are commercially available, e.g., from American Type Culture Collection, e.g., OKT3 (ATCC CRL 8001). An antibody can be administered for an appropriate time, e.g., at least 7 days, e.g., at least 10 days, e.g., at least 30 days, to inhibit rejection of cultured DP cells following transplantation. Antibodies can be administered intravenously in a pharmaceutically acceptable carrier, e.g., saline solution.

In some embodiments, the subject is treated, topically and/or systematically, with a hair growth promoting substance before, at the same time as, and/or after the transplantation of cells to enhance hair growth. Suitable hair growth promoting substances can include, e.g., minoxidil, cyclosporin, and natural or synthetic steroid hormones and their enhancers and antagonists, e.g., anti-androgens, all of which are commercially available.

B. Terminal Hair Induction Another embodiment provides a method for inducing vellus hair to become terminal hair. Vellus hair is the fine, non-pigmented hair (peach fuzz) that covers the body of children and adults. Terminal hair is developed hair, which is generally longer, coarser, thicker and darker than the shorter and finer vellus hair. As described above there is a morphogenetic switch of terminal to vellus hair follicles in the manifestation of male pattern baldness.

In one embodiment, dermal cells with increased trichogenic ability are injected into the skin as described above. The dermal cells are obtained as described above and are typically autologous cells. The cells are injected into or adjacent to vellus hair or vellus hair follicles. Multiple injections of dermal cells may be delivered to an area of skin containing vellus hair to induce as many vellus hair follicles as possible to become terminal hair follicles. It will be appreciated that the number of injections and volume of cells to be injected can be routinely developed by one of skill in the art.

In another embodiment, dermal cells with increased trichogenic activity are injected into skin in an amount effective to induce formation of hair follicles and to induce vellus hair follicles to become terminal hair follicles.

Unless defined otherwise, all technical and scientific terms used herein have the same meanings as commonly understood by one of skill in the art to which the disclosed invention belongs. Publications cited herein and the materials for which they are cited are specifically incorporated by reference.

### EXAMPLES

### Example 1

### Stimulation of the Sonic Hedgehog Pathway

Cell Culture

Cell dissociation can be obtained using any known procedure, including treatment with enzymes such as trypsin and collagenase, or by using physical methods of dissociation such as with a blunt instrument or by mincing with a scalpel to a allow outgrowth of specific cell types from a tissue.

Dissociated cells can be placed into any known culture medium capable of supporting cell growth, including MEM, DMEM, and RPMI, F-12, containing supplements which are required for cellular metabolism such as glutamine and other amino acids, vitamins, minerals and useful proteins such as transferrin. Medium may also contain antibiotics to prevent contamination with yeast, bacteria and fungi such as penicillin, streptomycin, and gentamicin. In some cases, the medium may contain serum derived from bovine, equine, or chicken. A particularly preferable medium for cells is a mixture of DMEM and F-12.

Shh pathway agonist(s) can be added with desired concentrations as an additive to the basal medium. Cells can be treated for Shh pathway agonist for 1, 2, 3, 4, 5, 6, 7 days or longer before harvest.

Results

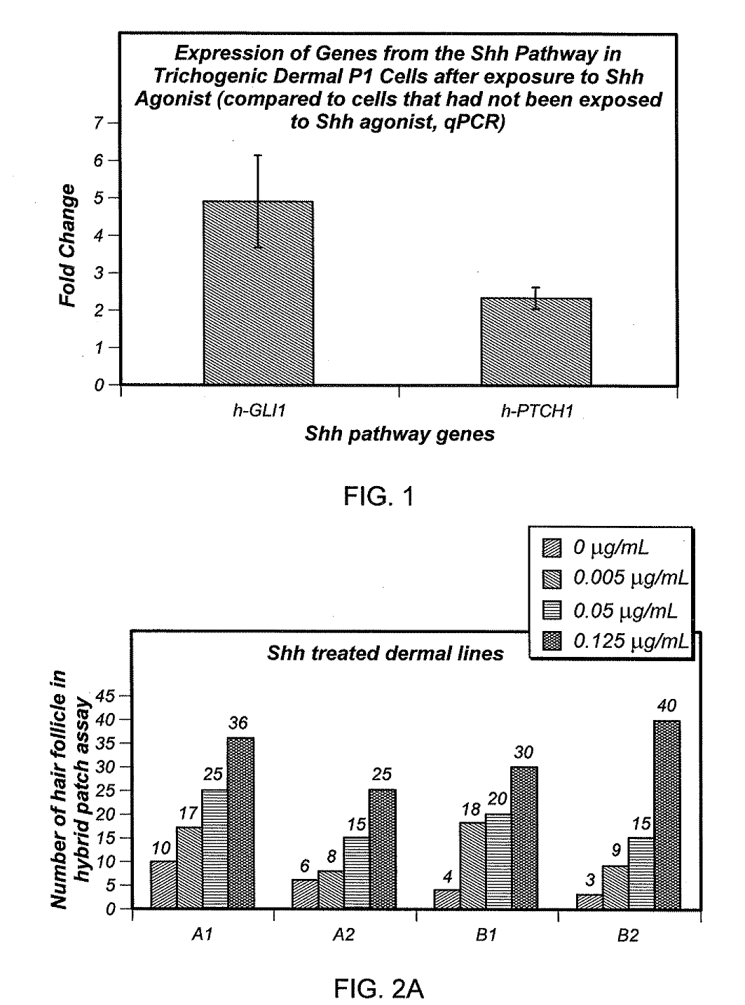

FIG. 1 demonstrates that the Shh pathway agonist compounds stimulate the transcription of components of the sonic hedgehog pathway. In a series of studies using dissociated and aggregated dermal cells from various patients, it was shown that exposing cells to a Shh pathway agonist stimulates the gene expression of various members of the Shh growth factor family (namely, Gli1 and Ptc); transcripts were detected by qPCR.

### Example 2

### Dose Response of the Compounds Used and the Bioassay

Aderans Hair Patch Assay™

Trichogenic activity of populations of dermal cells was determined by the Aderans Hair Patch Assay™ (Zheng, Y., J Invest Dermatol, 124: 867-876 (2005)). In this assay dissociated dermal and epidermal cells are implanted into the dermis or the subcutis of an immunoincompetent mouse. Using mouse newborn skin cells, new hair follicles typically form in this assay within 8 to 10 days. The newly formed follicle manifests normal hair shafts, mature sebaceous glands, and a natural hair cycle. Although normal cycling hair follicles are formed in this assay, the assay primarily measures the ability of cells or combinations of cells to form new follicles. In the classical Patch assay mouse neonatal dermal cells were assayed in conjunction with mouse neonatal epidermal cells. In a modification of that assay human adult dermal cells are assayed in the presence of mouse newborn epidermal cells.

Results

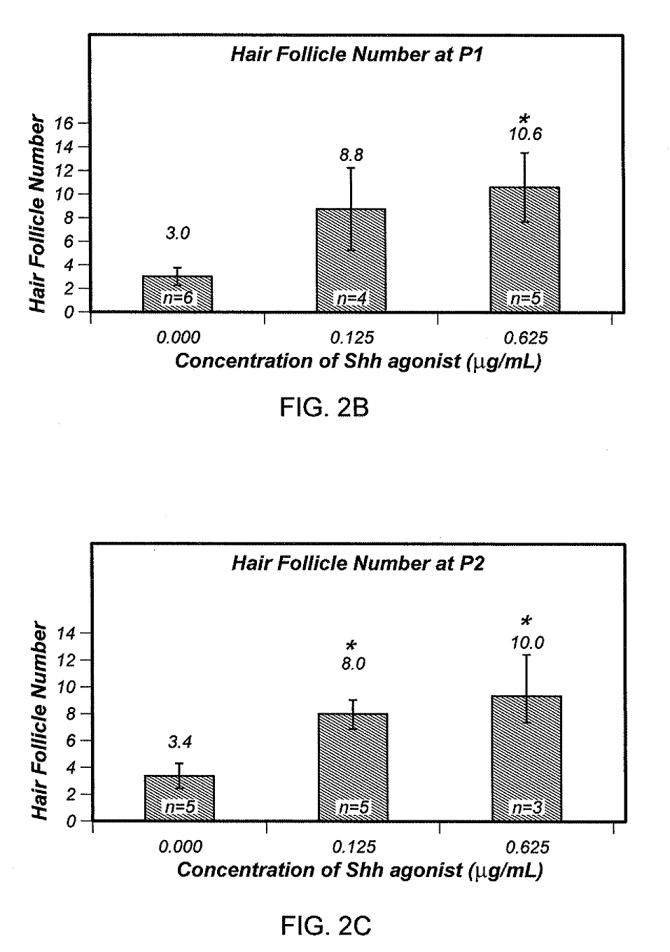

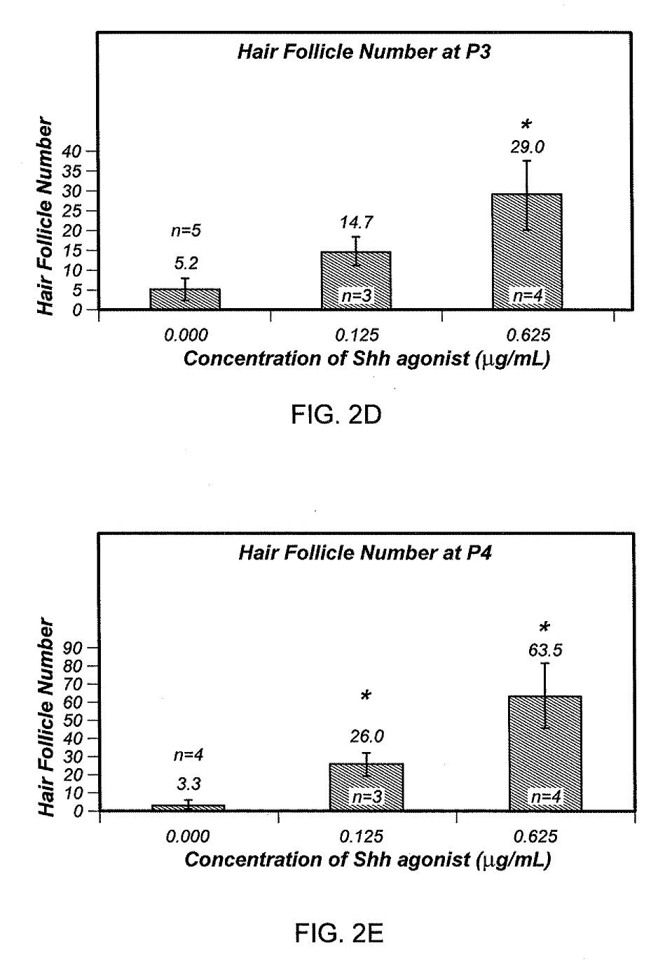

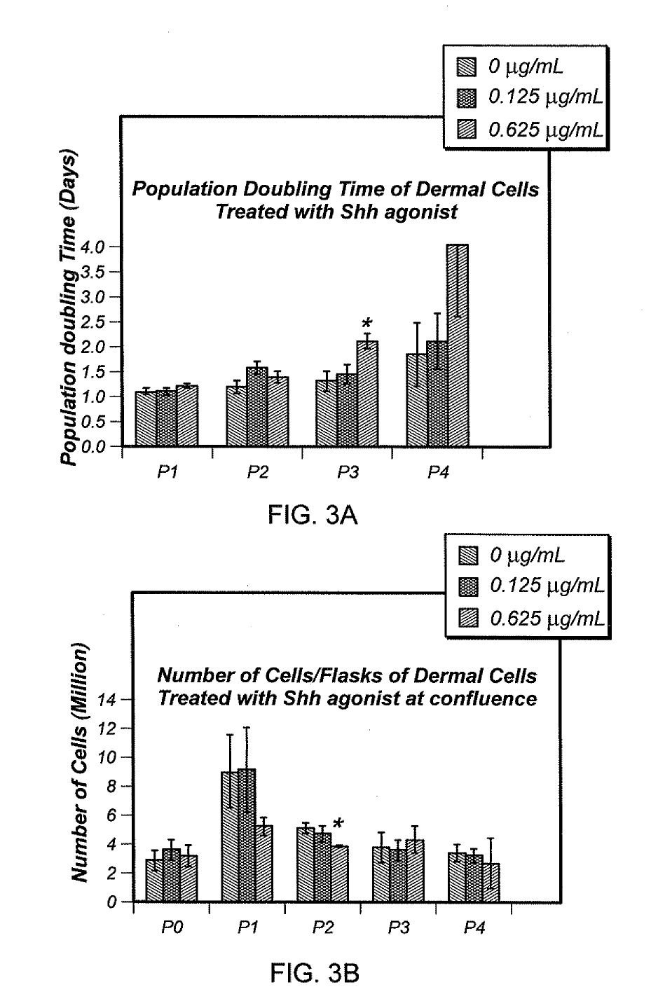

In the initial study four different concentrations of one of the Curls, Inc. Shh pathway agonist (agonist A-CUR-0201365) were used, 0, 0.005, 0.05 and 0.125 ug/ml (FIG. 2A). In the second study the dose was further increased to 0.625 ug/ml (FIG. 2B). Cells grown in medium containing Shh pathway agonist A (CUR-0201365) produced more hair follicles in the patch assay (per million cells injected) compared to non-treated cells. Furthermore, a dose-response relationship was observed with increasing effect (hair follicle number in patch assay) with increasing Shh pathway agonist concentration in the medium. Shh agonist treatment prolonged cell trichogenicity to later passages. With passage (P1 through P4) in the absence of the agonist, trichogenicity diminished while in the highest concentration the activity remained at a high level (FIG. 2B). Although the activity does not appear to flatten out, in the higher concentrations cell growth rate decreases (FIG. 3). The optimal concentration for agonist A would appear to be less than 0.625 μg/ml but greater than 0.125 μg/ml. The number of hair follicles formed in the hair patch assay is greater when the cells are grown in the presence of the Shh pathway agonist.

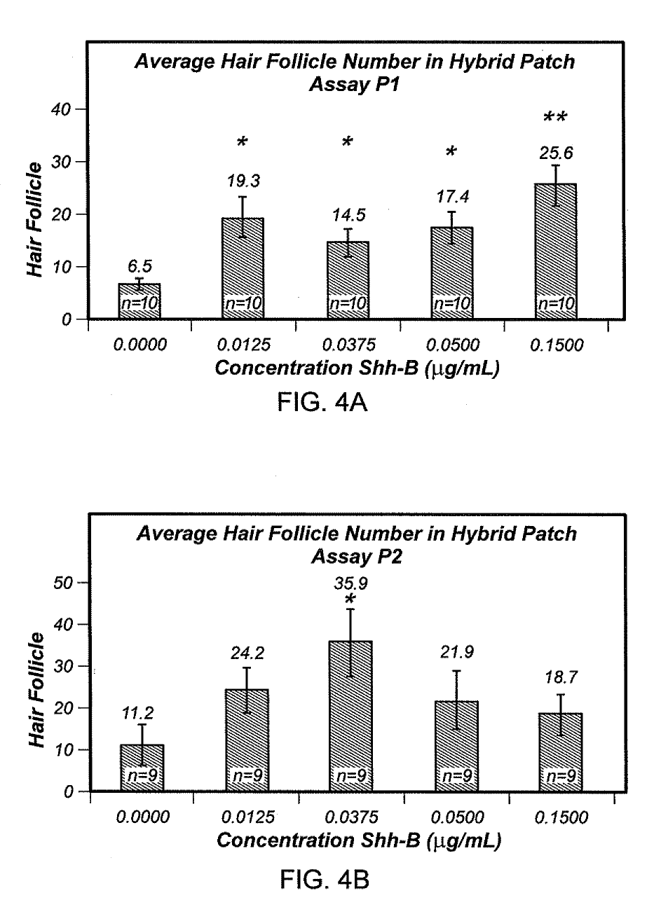

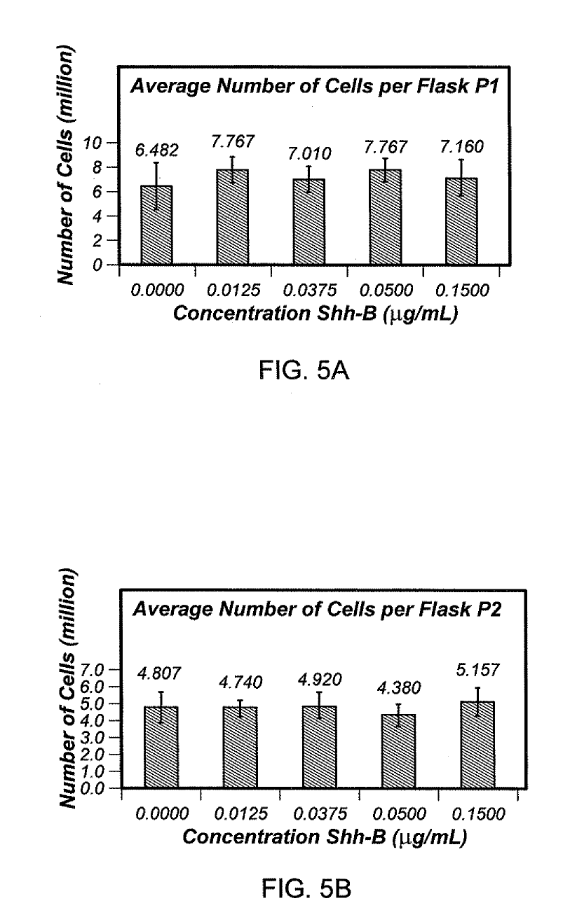

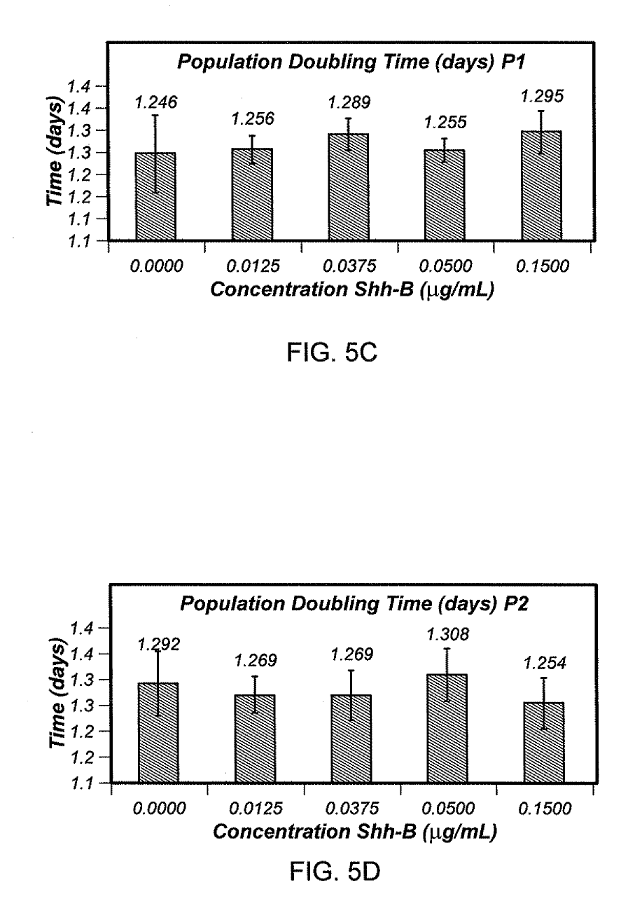

A second compound (agonist B, CUR-0236715) was tested. Like the first compound the treated cells supported increased hair follicle number in all patch assays with various agonist concentrations. All the concentrations showed significant difference in hair number compared to that of the non-treated control at P1. The middle concentration, 0.0375 μg/ml, was optimal for hair follicle formation (FIG. 4B) at P2. This second agonist (B) did not show any adverse effect on cell growth or yield in all the concentrations tested (FIG. 5).

### Example 3

### Cell Inductivity with Short-Term Treatment

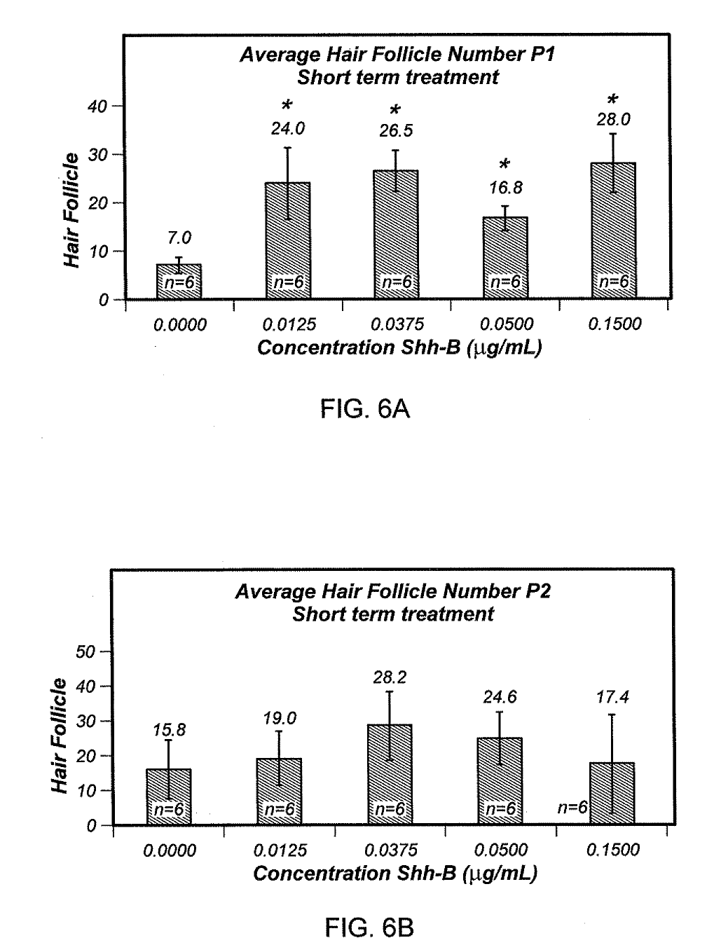

To investigate if Shh pathway agonist can increase cell inductivity with short-term treatment (7 days before harvest); 3 patient samples were tested using agonist B. For each sample, the cells were treated with 0, 0.0125, 0.0375, 0.05 and 0.15 ug/ml of Shh pathway agonist B at the passage P1 or P2. P1 and P2 cells were then harvested after approximately 7 days and analyzed in the hybrid patch assay. Similar results were seen in these short-term treated samples (FIGS. 6A and 6B), where all Shh pathway agonist B treated cells gave higher hair follicle numbers compared to the control. In this study the middle concentration (0.0375 μg/ml) proved to be the optimal for P1 and P2 cells. The middle concentration of Shh pathway agonist B increased hair follicle number in a short term treatment (7 days). Cells were treated for that 7 day period only, but not treated prior to that. The continuous treatment results are shown in FIG. 4. The short term results were comparable to that of continuous treatment.

### Example 4

### SHH Agonists on Human Fetal Cells and Mouse Cells

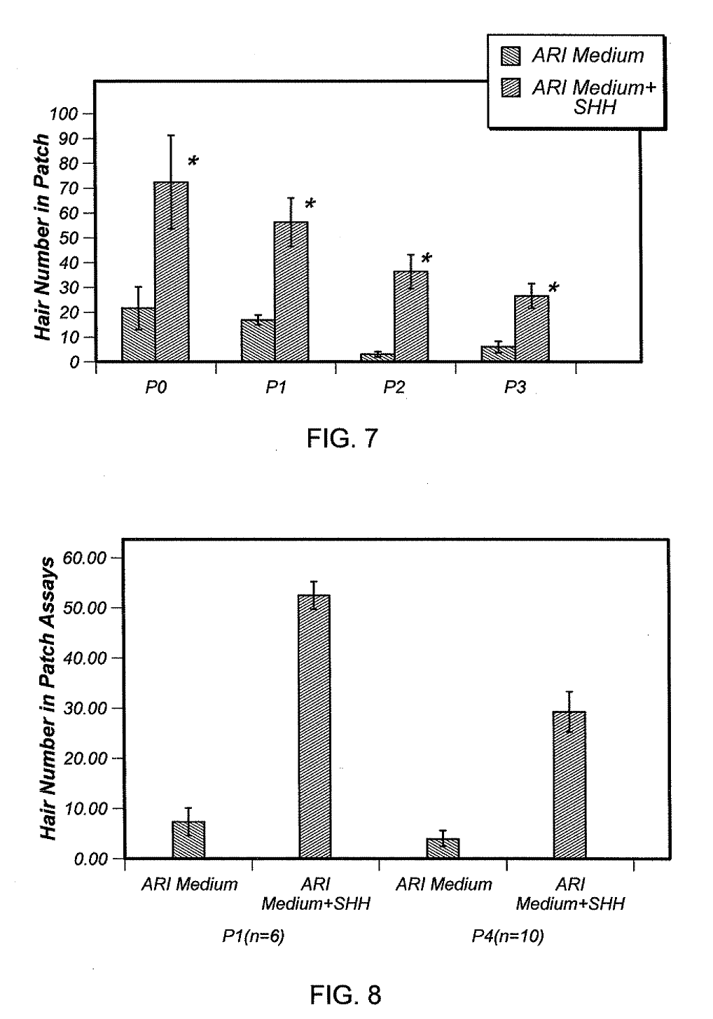

We have recently extended our study of the impact of SHH pathway agonist B (CUR-0236715) on hair inducing activity from human adult cells to human fetal cells, as well as mouse cells. Treating human fetal cells with SHH pathway agonist B (37.5 ng/ml) at P0 resulted in more than 3 fold increase in hair number in the Aderans Hair Patch Assay™ (FIG. 7), the fold change is very similar to that in the adult cells. The increase of hair number by SHH treatment extended to later passages (P3) when cells were continuously cultured in the presence of SHH pathway agonist (FIG. 7).

In another experiment, cells were initially cultured in medium without SHH pathway agonist. The SHH pathway agonist CUR-0236715 was only added to the culture at the beginning of a particular passage, and lasted for that passage only (P1 and P4 as shown in FIG. 8). Adding SHH pathway agonist B (CUR-0236715) at these later passages can still increase fetal cell trichogenicity. As shown in FIG. 2, when cells were only exposed the Shh pathway agonist B at P1 or P4, the number of hair follicles formed is about 5 fold higher than the no SHH control cells, and is also equivalent to the number of hairs formed by cells that were consistently treated by the agonist throughout the culture (FIG. 7).

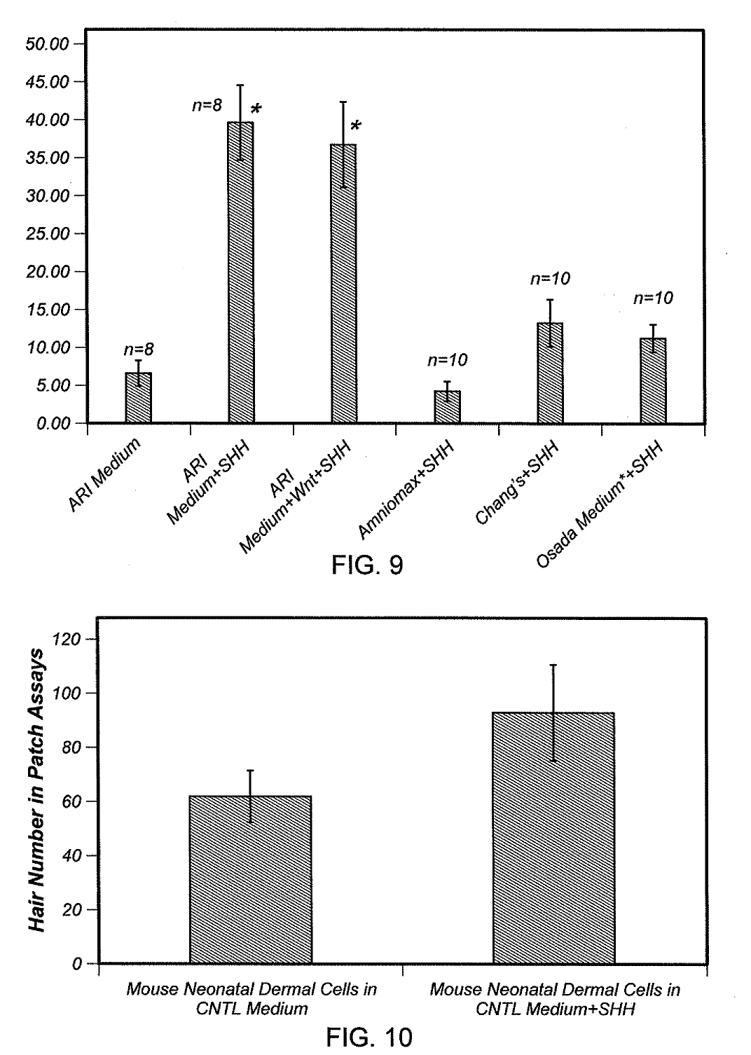

It is noteworthy that the trichogenicity enhancing effect of the SHH pathway agonist B may be medium dependent. As shown in FIG. 9 when the agonist is added to cell culture medium it resulted in increase of hair number, while in other commercially available media such as Amniomax and Chang's media (invitrogen, CA), and culture medium used by Osada, A., et al., Tissue Eng, 13(5):975-82 (2007) tested the effect is not significant.

In addition to cultured human cells, the SHH pathway agonist B was added to mouse neonatal dermal cell culture. Preliminary data showed that mouse cells treated with SHH pathway agonist also had increased activity as indicated by the number of hair follicles formed in patch assay (FIG. 10). This result showed that the trichogenicity enhancing effect of the SHH agonist is not limited to human cells. In addition to increase in hair number, the size of hair follicles formed by SHH agonist treated cells was also increased significantly compared to the size of hair follicles formed by non-treated cells.

Unless defined otherwise, all technical and scientific terms used herein have the same meanings as commonly understood by one of skill in the art to which the disclosed invention belongs.

Those skilled in the art will recognize, or be able to ascertain using no more than routine experimentation, many equivalents to the specific embodiments of the invention described herein. Such equivalents are intended to be encompassed by the following claims.

1. A method for increasing trichogenicity of cells in culture comprising
culturing dissociated mammalian dermal cells in vitro in the presence of an effective amount of a sonic hedgehog pathway agonist to increase the trichogenicity of the dissociated mammalian dermal cells compared to untreated dissociated mammalian dermal cells,
wherein the sonic hedgehog pathway agonist is defined by the following formula

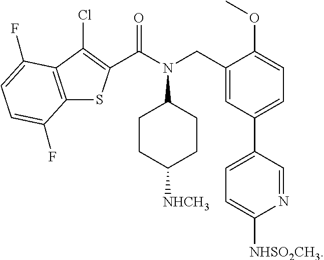

2. (canceled)

3. The method of claim 1 wherein the trichogenicity is determined using a patch assay.

4. The method of claim 1 wherein the mammalian cells are human.

5. The method of claim 1 wherein the dissociated mammalian dermal cells are cultured in the presence of an effective amount of the sonic hedgehog pathway agonist for at least 1, 2, 3, 4, 5, 6, 7 or more days.

6. A method for treating hair loss comprising
implanting the dissociated mammalian cells obtained from claim 1 into skin of a subject in need thereof in an amount effective to form a hair follicle.

7. An isolated population of mammalian cells obtained by claim 1.

8. A method of prolonging trichogenicity of dermal cells in culture comprising
dissociating dermal cells from a skin explant;
culturing the dissociated dermal cells in the presence of an effective amount of a sonic hedgehog pathway agonist to maintain the trichogenicity of the dissociated cells compared to untreated cells to at least the second passage;
injecting the cells into the skin of a subject in an amount effective to induce formation of a hair follicle or to reverse hair miniaturization.

9. The method of claim 8 wherein the dissociated dermal cells maintain trichogenicity to at least the third passage compared to untreated cells as determined by hair patch assay.

10. The method of claim 8 wherein the dissociated dermal cells maintain trichogenicity to at least the fourth passage compared to untreated cells as determined by hair patch assay.

11. A method for inducing hair follicle formation in a subject comprising
dissociating dermal cells from an explant from the subject;
culturing the dissociated dermal cells in the presence of an effective amount of a sonic hedgehog pathway agonist to increase the trichogenicity of the dissociated dermal cells compared to untreated dissociated dermal cells;
harvesting the dissociated dermal cells; and
injecting an effective amount of the harvested dissociated dermal cells into the subject to form a hair follicle or to reverse hair miniaturization,
wherein the sonic hedgehog pathway agonist is defined by the following formula

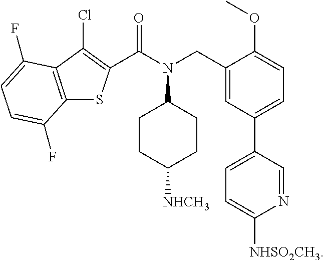

12. The method of claim 8, wherein the sonic hedgehog pathway agonist is defined by the following formula [chemical structure]
